(19) United States
(12) Patent Application Publication
(10) Pub. No.: US 2022/0257818 A1
(43) Pub. Date: Aug. 18, 2022

(54) AUTONOMOUS MOBILE DISINFECTING AND DEODORIZING SYSTEM AND METHOD

(71) Applicants: Petthannan Rajasekar, Chennai (IN); Kumaresh Krishnamoorthy, Bangalore (IN)

(72) Inventors: Petthannan Rajasekar, Chennai (IN); Kumaresh Krishnamoorthy, Bangalore (IN)

(21) Appl. No.: 17/273,893

(22) PCT Filed: Oct. 23, 2020
(86) PCT No.: PCT/IN2020/050900

§ 371 Date: Mar. 5, 2021

(30) Foreign Application Priority Data
Oct. 23, 2019 (IN) 2019 1043173

(51) Int. Cl. A61L 2/24; A61L 2/10; A61L 2/20; A61L 9/20; A61L 9/04; A61L 9/12

(52) U.S. Cl. CPC A61L 2/24; A61L 2/10; A61L 2/202; A61L 9/20; A61L 9/046; A61L 2209/12; A61L 2202/16; A61L 2202/14; A61L 2202/11; A61L 2202/25; A61L 2209/111; A61L 9/12

(57) ABSTRACT

The various embodiments of herein provide a system and a method for autonomous and contextual disinfection and deodorization of an environment. The system is also configured to provide a constant energy radiation of UV. The sweeping motors in the system enable covering maximum area for disinfection without moving the whole system. The system is configured with Ultraviolet (UV) radiation unit, which provides a constant radiation of energy for pre-set time duration. The UV radiation unit comprises a plurality of reflective mirror surfaces that are designed to rotate about their axis. The system is configured to identify, in real-time, the requirement of the disinfection and accordingly move to the location to disinfect and/or rotate the reflective mirror surfaces to enable effective disinfection of the environment. The system is also configured with ozone generator to provide deodorization of an environment.

### CROSS-REFERENCE TO RELATED APPLICATIONS

The embodiments herein claim the priority of the Indian Provisional Patent Application filed on Oct. 23, 2019, with the number 201941043173 and titled, “AN AUTONOMOUS MOBILE DISINFECTING AND DEODORIZING SYSTEM AND METHOD”, and subsequently filed as a PCT application on Oct. 23, 2020, the contents of which are incorporated herein by the way of reference.

### BACKGROUND

### Description of the Related Art

The embodiments herein are generally related to a system and method for disinfection and deodorization of an environment. The embodiments herein are particularly related to a system and a method for autonomous and contextual disinfection and deodorization of an environment. The embodiments herein are more particularly related to an autonomous mobile system and method for enabling constant energy radiation to provide disinfection and deodorization of an environment.

### Description of the Related Art

The prevention of contamination and infections is one of the key factors to lead a healthy life. From an individual perspective, humans are taught and equipped to take proper care to safeguard themselves from infections. However, the different environments a person is exposed to during everyday life pose a serious challenge to the health of the person.

Since hospitals are a converging point for people of different illness and diseases, a hospital environment is perhaps the most difficult to disinfect. Some studies suggest that about 10% of people visiting a hospital contract an infection from the environment. This is a serious problem and proper disinfection systems are needed to keep infecting environments such as hospitals safe.

Currently, Ultraviolet (UV) based disinfection systems are available to be used in indoor environments. However, these systems are stationary or manually maneuverable. Also, current systems are moved from location to location for disinfection of environment and surfaces. They are also not designed to enable constant energy radiation method of disinfection.

Hence, there exists a need for a system and method to enable autonomous and contextual disinfection of indoor environments. There is also a need for a system and method to effectively utilize constant energy radiation technique for disinfection of surfaces in an indoor environment. There is also a need for a system to provide deodorization of indoor environments through a mobile ozone generator.

The above-mentioned shortcomings, disadvantages and problems are addressed herein, and which will be understood by reading and studying the following specification.

### OBJECT OF THE EMBODIMENTS HEREIN

The primary object of the embodiments herein is to provide a system and a method for autonomous and contextual disinfection and deodorization of an environment.

Another object of the embodiments herein is to provide an autonomous mobile system and method for enabling constant energy radiation to provide disinfection and deodorization of an environment.

Yet another object of the embodiments herein is to provide a system and method to enable autonomous and contextual disinfection of indoor environments.

Yet another object of the embodiments herein is to provide a system and method to effectively utilize constant energy radiation technique for disinfection of surfaces in an indoor environment.

Yet another object of the embodiments herein is to provide a system to provide deodorization of indoor environments through a mobile ozone generator.

These and other objects and advantages of the embodiments herein will become readily apparent from the following summary and the detailed description taken in conjunction with the accompanying drawings.

### SUMMARY

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The other objects and advantages of the embodiments herein will become readily apparent from the following description taken in conjunction with the accompanying drawings.

The various embodiments herein provide a system and a method for autonomous and contextual disinfection and deodorization of an environment. The embodiments also provide an autonomous mobile system and method for enabling constant energy radiation to provide disinfection and deodorization of an environment.

According to one embodiment herein, an autonomous and mobile system is provided for disinfection and deodorization of an environment. The system is a self-navigating mobile system that is configured to navigate an indoor structure. The system is configured with simultaneous localization and mapping capabilities to explore an indoor structure and build a map of the structure, along with a preset map of the structure. The system is also configured with Ultraviolet (UV) radiation unit, which provides a constant radiation of energy for preset time duration. The UV radiation unit comprises a plurality of reflective mirror surfaces that are designed to rotate about their axis. The system is configured to identify, in real-time, the requirement of the disinfection and accordingly move to the location to disinfect and/or rotate the reflective mirror surfaces to enable effective disinfection of the environment. The system is also configured with ozone generator to provide deodorization of an environment.

According to one embodiment herein, a system is provided for autonomous and contextual disinfection and deodorization of an environment. The system comprises a wheeled platform that comprises a Ultraviolet radiation unit mounted on the platform. The system also comprises a control module, wherein the control module is equipped with digital storage and communication capabilities. The system includes a sensor module that comprises proximity sensors, imaging sensors and a plurality of inertial sensors. The Ultraviolet radiation unit further comprises a plurality of Ultraviolet generators, a plurality of reflectors and motors to enable a rotational motion of the Ultraviolet generators and the reflectors.

According to one embodiment herein, a sweeping beam concentrator is provided. The sweeping beam concentrator comprises polished mirror surfaces of reflection of UV radiation and refocusing the radiation in a desired direction and location. The sweeping beam concentrator arrangement is designed to rotate at variable angles, thus enabling the sweeping beam concentrator to have a moving focus point to impart maximum radiation intensity.

According to one embodiment herein, a plurality of modes and methods of operation is provided for the autonomous and contextual system for disinfection and deodorization of an environment. The modes and methods of operation include manual mode, remote controlled mode, semi-autonomous mode, and completely autonomous mode.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a side view of a system for enabling autonomous and mobile disinfection, according to one embodiment herein.

FIG. 2 illustrates a front view of a system for enabling autonomous and mobile disinfection, according to one embodiment herein.

FIG. 3 illustrates a zoomed in front view of a system for enabling autonomous and mobile disinfection, according to one embodiment herein.

FIG. 4 illustrates a front view of an Ultraviolet radiation unit, according to one embodiment herein.

FIG. 5 illustrates an arrangement of motors enabling rotation of Ultraviolet radiation unit, according to one embodiment herein.

Although the specific features of the embodiments herein are shown in some drawings and not in others. This is done for convenience only as each feature may be combined with any or all of the other features in accordance with the embodiment herein.

### DETAILED DESCRIPTION OF THE EMBODIMENTS HEREIN

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that other changes may be made without departing from the scope of the embodiments. The following detailed description is therefore not to be taken in a limiting sense.

The various embodiments herein provide a system and a method for autonomous and contextual disinfection and deodorization of an environment. The embodiments herein also provide an autonomous mobile system and method for enabling constant energy radiation to provide disinfection and deodorization of an environment.

According to one embodiment herein, a system is provided for an autonomous mobile apparatus that enables disinfection and deodorization of an indoor environment through Ultraviolet (UV) radiation. The system comprises an Ultraviolet radiation unit, a wheeled platform, a control module, a sensor module, and an ozone generator. The Ultraviolet radiation unit further comprises a plurality of Ultraviolet generators, a plurality of reflectors, a plurality of motors and a sweeping beam concentrator. The wheeled platform acts as a base structure on which the Ultraviolet radiation unit is mechanically mounted and designed to enable mobility to the system. The ozone generator is configured for deodorization of an environment in which the system is placed.

According to one embodiment herein, the plurality of Ultraviolet generators are sources of Ultraviolet radiation. The Ultraviolet generators are cylindrically shaped structures designed to emit Ultraviolet radiation through the entirety of their cylindrical surface. The Ultraviolet generators are configured to provide a constant radiation of energy for a pre-set duration of time. The reflectors are mechanically coupled to the Ultraviolet generators such that each Ultraviolet generator and reflector form an assembly, and each of the assemblies is designed to rotate about its axis.

According to one embodiment herein, the motors are configured to mechanically couple with the Ultraviolet generator and reflector assembly such that the rotational motion of the motor enables the assembly to rotate about its axis. The rotating assembly irradiates the surroundings of the system with a uniform dosage of Ultraviolet radiation.

According to one embodiment herein, the sweeping beam concentrator comprises a plurality of polished mirror surfaces for reflection of Ultraviolet radiation and refocusing the radiation in a desired direction and location. The sweeping beam concentrator arrangement is designed to rotate in a plurality of angles to enable the sweeping beam concentrator to have a moving focus point to impart maximum and uniform radiation intensity on all the surfaces irradiated.

According to one embodiment herein, the control module is equipped with digital storage, computing and digital communication capabilities. The control module is configured to control the activation, deactivation and dosage of the Ultraviolet radiation emitted by the Ultraviolet radiation generators. The control module is also configured to control the rotation of the motors and sweeping beam concentrator. The control module is also configured to communicably couple with a remote computing device to enable a remote operation of all the functionalities of the Ultraviolet radiation unit and the control module through the remote computing device.

According to one embodiment herein, sensor module comprises proximity sensors, imaging sensors, audio-visual sensors and a plurality of inertial sensors. The sensor module is configured to communicate the sensor readings to the control module. The control module is configured to process the sensor readings and contextually change the operation of the Ultraviolet radiation unit.

According to one embodiment herein, the system for providing an autonomous mobile apparatus that enables disinfection and deodorization of an indoor environment through Ultraviolet (UV) radiation is configured to be operated in a plurality of different modes, including manual mode, remote controlled mode, semi-autonomous and fully autonomous mode.

According to one embodiment herein, the wheeled platform is configured to be connected to a plurality of motors that are controlled by the control module when the system is set to operate in remote controlled, semi-autonomous or fully autonomous modes, such that the motion of the system is controlled by the control module.

According to one embodiment herein, the dosage of Ultraviolet radiation and time required for disinfection of a plurality of microorganisms and pathogens are readily preconfigured in the control module. The control module is configured to vary the intensity of Ultraviolet radiation from 0% to 100% in a programmable manner. The optimal dosage required for disinfecting a particular microorganism or pathogen is identified by a human user in the manual mode of operation and the control module in full autonomous mode of operation.

According to one embodiment herein, an autonomous and mobile system is provided for disinfection and deodorization of an environment. The system is a self-navigating mobile system that is configured to navigate an indoor structure. The system is configured with simultaneous localization and mapping capabilities to explore an indoor structure and build a map of the structure, along with a preset map of the structure. The system is configured with Ultraviolet (UV) radiation unit, which provides a constant radiation of energy for preset time duration. The UV radiation unit comprises a plurality of reflective mirror surfaces that are designed to rotate about their axis. The system is configured to identify, in real-time, the requirement of the disinfection and accordingly move to the location to disinfect and/or rotate the reflective mirror surfaces to enable effective disinfection of the environment. The system is also configured with ozone generator to provide deodorization of an environment.

According to one embodiment herein, a system is provided for autonomous and contextual disinfection and deodorization of an environment. The system comprises a wheeled platform that comprises an Ultraviolet radiation unit mounted on the platform. The system also comprises a control module, wherein the control module is equipped with digital storage and communication capabilities. The system includes a sensor module that comprises proximity sensors, imaging sensors and a plurality of inertial sensors. The Ultraviolet radiation unit further comprises a plurality of Ultraviolet generators, a plurality of reflectors and motors to enable a rotational motion of the Ultraviolet generators and the reflectors.

According to one embodiment herein, a sweeping beam concentrator is provided. The sweeping beam concentrator comprises polished mirror surfaces of reflection of UV radiation and refocusing the radiation in a desired direction and location. The sweeping beam concentrator arrangement is designed to rotate at variable angles, thus enabling the sweeping beam concentrator to have a moving focus point to impart maximum radiation intensity.

According to one embodiment herein, a plurality of modes and methods of operation is provided for the autonomous and contextual system for disinfection and deodorization of an environment. The modes and methods of operation include manual mode, remote controlled mode, semi-autonomous mode and completely autonomous mode.

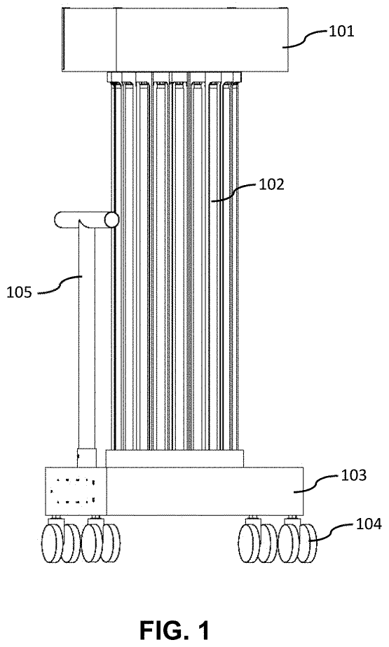

FIG. 1 illustrates a side view of a system for enabling autonomous and mobile disinfection. The system comprises a control module 101, a UV radiation unit 102, a platform 103, wheels 104 and a handle 105.

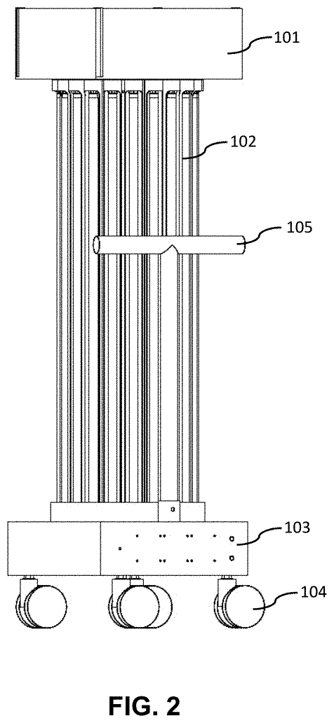

FIG. 2 illustrates a front view of a system for enabling autonomous and mobile disinfection.

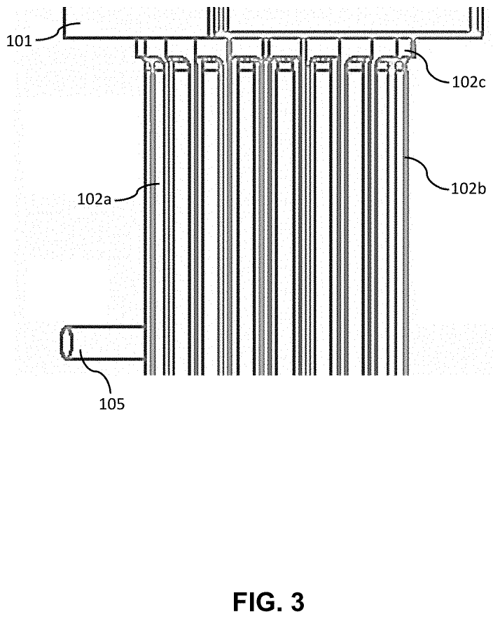

FIG. 3 illustrates a zoomed in front view of a system for enabling autonomous and mobile disinfection. The UV radiation unit 102 further comprises a UV generator module 102a, reflector module 102b and sweeping motors 102c.

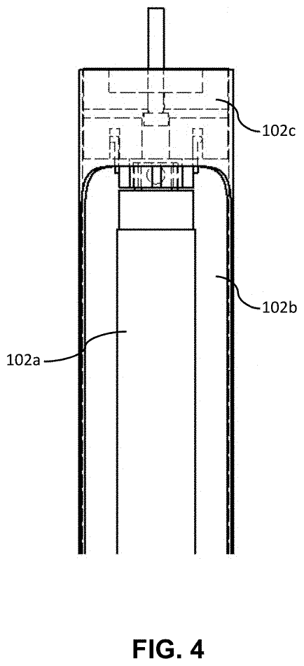

FIG. 4 illustrates a front view of an Ultraviolet radiation unit.

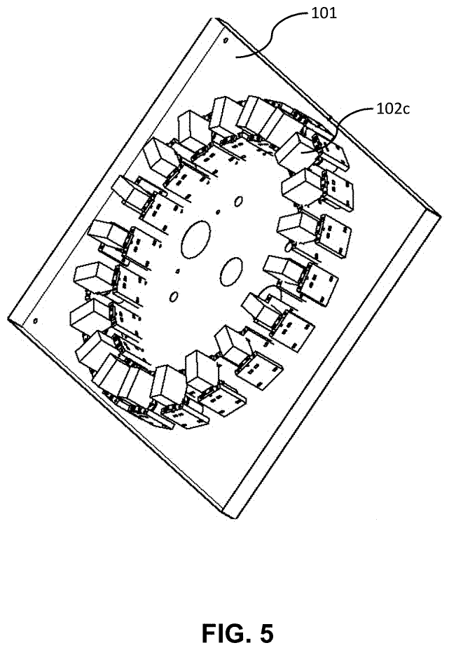

FIG. 5 illustrates an arrangement of motors enabling rotation and sweeping motion of Ultraviolet radiation unit.

The various embodiments herein provide a system and a method for autonomous and contextual disinfection and deodorization of an environment. The embodiments also provide an autonomous mobile system and method for enabling constant energy radiation to provide disinfection and deodorization of an environment. The system is provided with capabilities to identify a requirement of radiation in an environment and contextually provide the same. The system is also configured to provide a constant energy radiation of UV, which provides an efficient and effective disinfection. The sweeping motors in the system enable covering maximum area for disinfection without moving the whole system.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the disclosure with modifications. However, all such modifications are deemed to be within the scope of the appended claims.

It is also to be understood that the following claims are intended to cover all of the generic and specific features of the embodiments described herein and all the statements of the scope of the embodiments which as a matter of language might be said to fall there between.

1. A system for providing an autonomous mobile apparatus that enables disinfection and deodorization of an indoor environment through Ultraviolet (UV) radiation, the system comprising:
an Ultraviolet radiation unit, wherein the Ultraviolet radiation unit further comprises a plurality of Ultraviolet generators, a plurality of reflectors, a plurality of motors and a sweeping beam concentrator;
a wheeled platform, wherein the wheeled platform acts as a base structure on which the Ultraviolet radiation unit is mechanically mounted and designed to enable mobility to the system;
a control module;
a sensor module; and
an ozone generator, wherein the ozone generator is configured for deodorization of an environment in which the system is placed.

2. The system according to claim 1, wherein the plurality of Ultraviolet generators are sources of Ultraviolet radiation, and wherein the Ultraviolet generators are cylindrically shaped structures designed to emit Ultraviolet radiation through the entirety of their cylindrical surface, and wherein the Ultraviolet generators are configured to provide a constant radiation of energy for a preset duration of time, and wherein the reflectors are mechanically coupled to the Ultraviolet generators such that each Ultraviolet generator and reflector form an assembly, and wherein each of the assemblies is designed to rotate about its axis.

3. The system according to claim 1, wherein the motors are configured to mechanically couple with the Ultraviolet generator and reflector assembly such that the rotational motion of the motor enables the assembly to rotate about its axis, and wherein the rotating assembly irradiates the surroundings of the system with a uniform dosage of Ultraviolet radiation.

4. The system according to claim 1, wherein the sweeping beam concentrator comprises a plurality of polished mirror surfaces for reflection of Ultraviolet radiation and refocusing the radiation in a desired direction and location, and wherein the sweeping beam concentrator arrangement is designed to rotate in a plurality of angles to enable the sweeping beam concentrator to have a moving focus point to impart maximum and uniform radiation intensity on all the surfaces irradiated.

5. The system according to claim 1, wherein the control module is equipped with digital storage, computing and digital communication capabilities, and wherein the control module is configured to control the activation, deactivation and dosage of the Ultraviolet radiation emitted by the Ultraviolet radiation generators, and wherein the control module is also configured to control the rotation of the motors and sweeping beam concentrator, and wherein the control module is also configured to communicably couple with a remote computing device to enable a remote operation of all the functionalities of the Ultraviolet radiation unit and the control module through the remote computing device.

6. The system according to claim 1, wherein sensor module comprises proximity sensors, imaging sensors, audio-visual sensors and a plurality of inertial sensors, and wherein the sensor module is configured to communicate the sensor readings to the control module, and wherein the control module is configured to process the sensor readings and contextually change the operation of the Ultraviolet radiation unit.

7. The system according to claim 1, wherein the system for providing an autonomous mobile apparatus that enables disinfection and deodorization of an indoor environment through Ultraviolet (UV) radiation is configured to be operated in a plurality of different modes, including manual mode, remote controlled mode, semi-autonomous and fully autonomous mode.

8. The system according to claim 1, wherein the wheeled platform is configured to be connected to a plurality of motors that are controlled by the control module when the system is set to operate in remote controlled, semi-autonomous or fully autonomous modes, such that the motion of the system is controlled by the control module.

9. The system according to claim 1, wherein the dosage of Ultraviolet radiation and time required for disinfection of a plurality of microorganisms and pathogens are readily preconfigured in the control module, and wherein the control module is configured to vary the intensity of Ultraviolet radiation from 0% to 100% in a programmable manner, and wherein the optimal dosage required for disinfecting a particular microorganism or pathogen is identified by a human user in the manual mode of operation and the control module in full autonomous mode of operation.
